(19) United States
(12) Patent Application Publication
(10) Pub. No.: US 2004/0082911 A1
(43) Pub. Date: Apr. 29, 2004

(54) Syringe having needle safely receiving structure

(76) Inventors: Bruce Tiu, Kaoshiung (TW); Ching Chao Hou, Kaoshiung (TW)

Correspondence Address: Bruce Tiu, P.O. Box 63-99, Taichung, 406, TW

(21) Appl. No.: 10/279,730

(22) Filed: Oct. 25, 2002

(51) Int. Cl. A61M 5/00

(52) U.S. Cl. 604/110

(57) ABSTRACT

A syringe includes a needle support received in a front portion of a housing and having a front tube for attaching a needle. A plunger includes a piston slidably received in the housing and a stem detachably secured to the piston. The piston may be secured to the needle support for pulling the needle into the housing after use. The needle support has one or more bulges for engaging with juts of the housing for limiting the needle support to rotate relative to the housing. The piston includes a rod and a pole for adequately forcing medicine out of the needle support.

### BACKGROUND OF THE INVENTION

1. Field of the Invention

The present invention relates to a syringe, and more particularly to a syringe including a needle safely receiving structure for safely receiving the needle after use and for preventing the syringe from being used again after use.

2. Description of the Prior Art

Typical syringes may be used to draw blood, or may be used to inject medicine or the like into human bodies. Due to the acquired immune deficiency syndrome (AIDS) and the other contagious diseases, the needles of the syringes, and even the syringes themselves, should not be used again and should be discarded right after use, for safety purposes.

U.S. Pat. No. 5,785,687 to Saito, U.S. Pat. No. 6,093,171 to Huang, U.S. Pat. No. 6,196,997 to Saito, and U.S. Pat. No. 6,368,306 to Koska disclose four of the typical syringes each of which also includes a piston rod that may be broken and disengaged from the piston after use. The needle may also be safely received in the syringe housing after use.

For retaining the needle in the syringe housing, an elastomeric cap or a needle support is required to be secured into the front end of the syringe housing. However, the needle support is normally rotatably secured in the syringe housing and retained in the syringe housing with such as a force-fitted engagement, or by the resilience force of the needle support, such that the needle support may be rotated relative to the syringe housing when threading or rotating the needle onto or into the needle support.

For retracting the needle backward into the syringe housing, the front end of the piston normally includes a spring latch for resiliently securing to the needle support, and thus for retracting the needle support and thus the needle into the syringe housing after use.

Accordingly, the front end of the piston is primarily provided for resiliently securing to the needle support with the spring latch, and may no longer be provided for attaching further members and for further deeply engaging into the needle support, and thus may not be used for completely forcing the residual medicine out of the needle support and out of the syringe housing after use.

Therefore, after use, the typical syringes may include a relatively larger amount of the residual medicine within the needle support, or may include a residual medicine greater than 0.075 ml which is the international standard requirement.

The present invention has arisen to mitigate and/or obviate the afore-described disadvantages of the conventional syringes.

### SUMMARY OF THE INVENTION

The primary objective of the present invention is to provide a syringe including a needle safely receiving structure for safely receiving the needle after use and for preventing the syringe from being used again after use.

The other objective of the present invention is to provide a syringe including a needle support that may be stably received within the syringe housing and may be prevented from rotating relative to the syringe housing, for allowing the needle to be easily threaded or rotated relative to the needle support and to be easily attached to the syringe housing.

The further objective of the present invention is to provide a syringe including a structure for allowing the medicine to be adequately forced or injected outward of the syringe housing and the needle support, and for allowing the amount of the residual medicine to be smaller than 0.075 ml.

In accordance with one aspect of the invention, there is provided a syringe comprising a housing including a front portion and a rear portion, a needle support received in the front portion of the housing, and including a front portion having a tube extended therefrom, and including a bore formed therein, a needle attached to the tube of the needle support and extendible outward of the housing, and a plunger including a piston slidably received in the housing and movable toward and away from the needle support, and including a stem detachably secured to the piston, for moving the piston toward and away from the needle support. The needle support includes a rear portion having at least one spring latch provided thereon, and a notch formed between the spring latch and the needle support, the piston includes a front portion having a peripheral rib laterally extended therefrom for moving over the spring latch and for engaging into the notch of the needle support, and for securing the piston to the needle support when the piston is forced toward the needle support, the needle support is movable into the housing by the plunger when the piston of the plunger is moved away from the front portion of the housing, and the housing includes at least one jut provided in the front portion thereof, the needle support includes at least one bulge extended therefrom for engaging with the jut of the housing, and for limiting a rotational movement between the needle support and the housing.

The jut of the housing includes a first end located at the front portion of the housing, and includes a second end directed toward the rear portion of the housing and sharper than the first end of the jut.

The needle support includes a rear portion, the bulge of the needle support includes a first end located at the front portion of the needle support, and includes a second end directed toward the rear portion of the needle support and sharper than the first end of the bulge.

The needle support includes a peripheral wall provided on the front portion thereof and provided and formed around the tube for forming a peripheral space between the tube and the peripheral wall, the jut is provided on the peripheral wall.

The peripheral wall includes at least one groove formed therein for increasing a resilience of the peripheral wall.

The housing includes an inner peripheral recess formed in the front portion thereof and defined by a peripheral rib, the needle support includes a peripheral swelling extended outwardly therefrom for moving over the peripheral rib and for engaging into the peripheral recess of the housing, and for detachably securing the needle support to the housing.

The needle support includes at least one projection extended from the peripheral swelling for engaging into the peripheral recess of the housing.

The needle support includes a rear portion having a chamber formed therein and communicating with the bore thereof, the piston includes a rod and a pole extended forwardly therefrom for engaging into the chamber and the bore of the needle support respectively.

The piston includes a rear portion having an extension extended therefrom, the stem includes a front portion having a channel formed therein for receiving the extension of the piston, and for defining a slot between the stem and the extension of the piston, and at least one coupling member formed and coupled between the stem and the extension of the piston, for detachably securing the stem to the piston.

The piston includes a head formed on the extension thereof, the stem includes an opening formed therein and communicating with the channel thereof for receiving the head, and includes at least one coupling member formed and coupled between the stem and the head of the piston.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is an exploded view of a syringe in accordance with the present invention;

FIG. 2 is a cross sectional view taken along lines 2-2 of FIG. 3;

FIG. 3 is a cross sectional view taken along lines 3-3 of FIG. 2;

FIG. 4 is an enlarged perspective view illustrating a needle support of the syringe; and

FIGS. 5, 6, 7 are cross sectional views illustrating the operation of the syringe.

### DETAILED DESCRIPTION OF THE PREFERRED EMBODIMENT

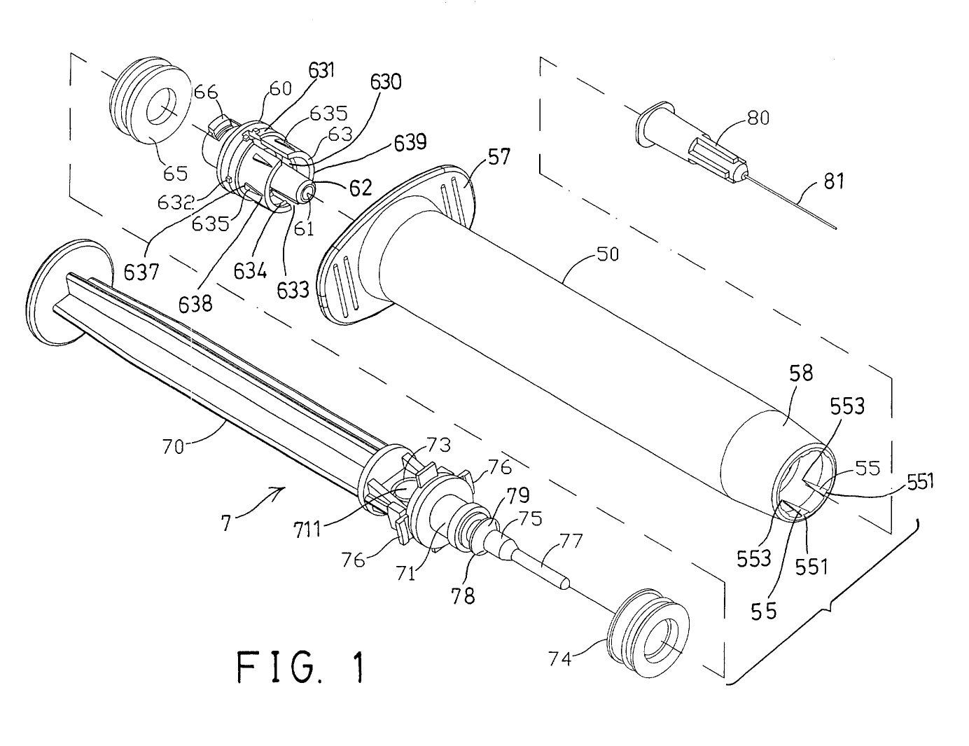

Referring to the drawings, and initially to FIGS. 1-4, a syringe in accordance with the present invention comprises a barrel or syringe housing 50 for receiving the blood to be drawn from the patient or for receiving the medicine or the other liquid or fluid, or the like to be injected into the patient.

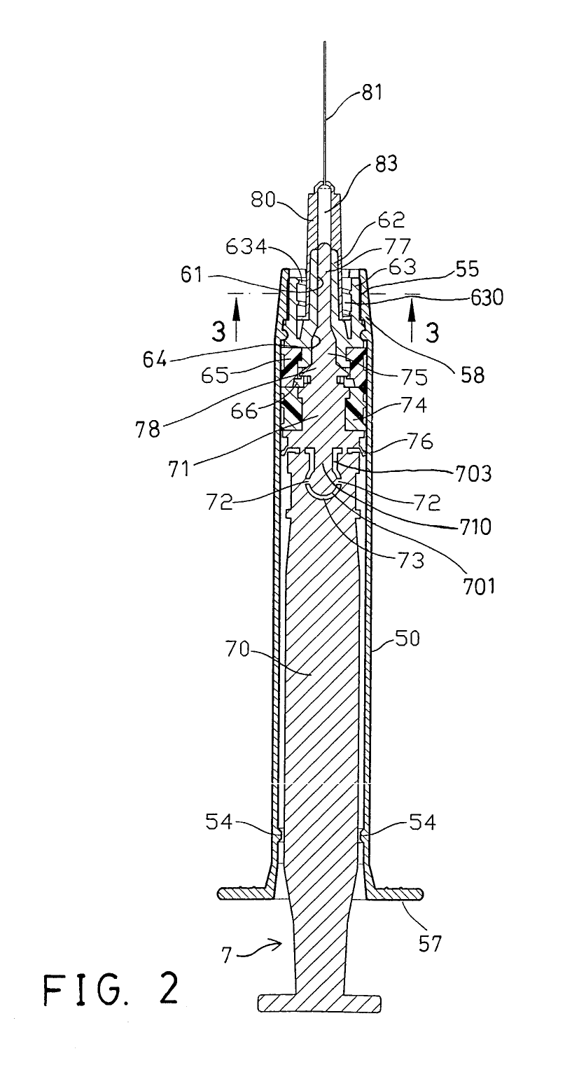

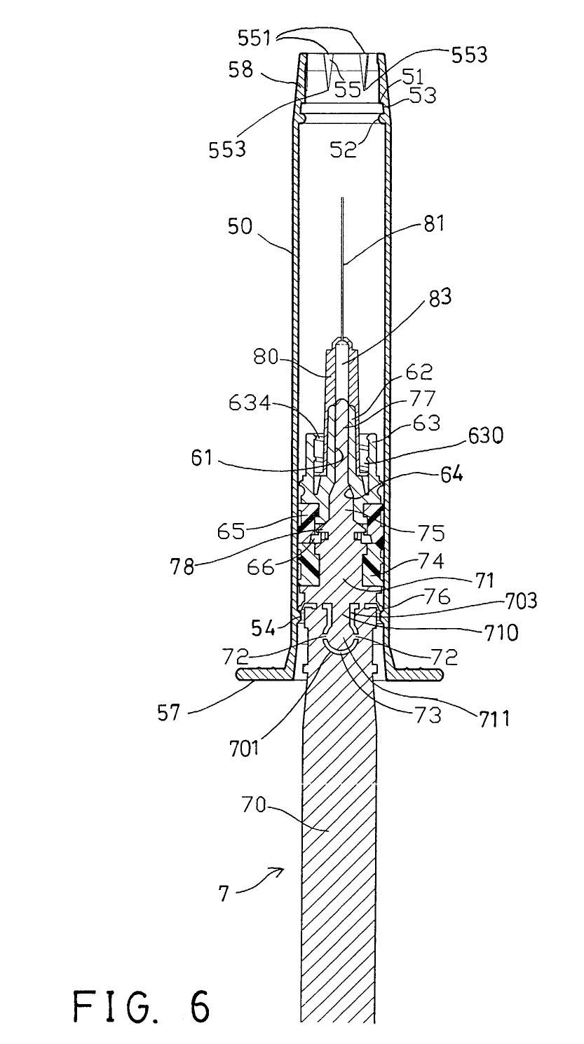

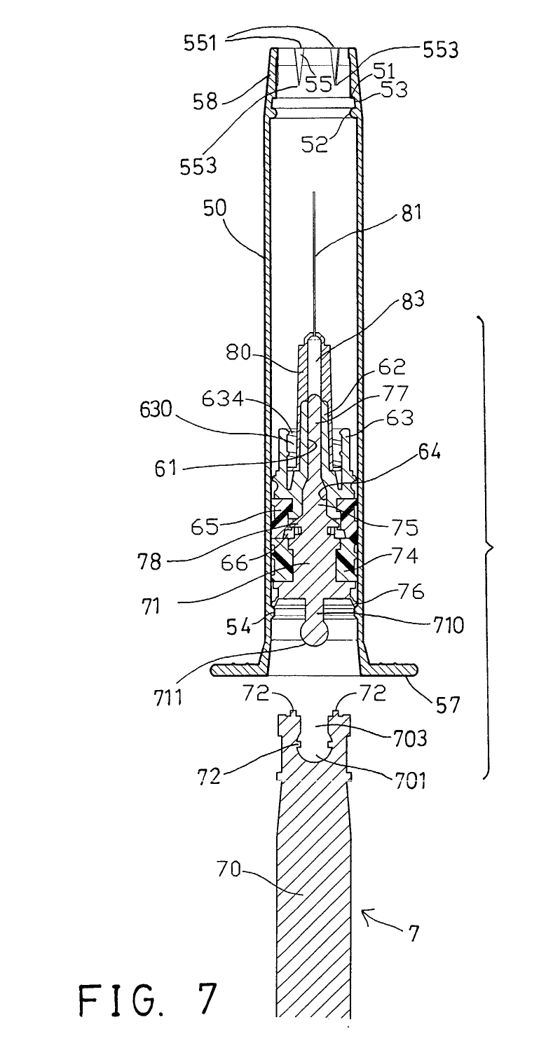

The syringe housing 50 includes an inner peripheral shoulder 51 and an inner peripheral rib 52 formed in the front portion thereof (FIGS. 6, 7) and a peripheral recess 53 formed between the peripheral shoulder 51 and the peripheral rib 52 thereof, and includes an inner peripheral flange 54 formed in the rear portion thereof (FIGS. 2, 5-7), and includes an enlarged panel 57 extended laterally outward from the rear portion thereof for being held or grasped by the users.

A needle support 60 is engaged in the front portion of the housing 50 and includes a peripheral swelling 631 extended radially and outwardly from the outer peripheral portion thereof for engaging over the peripheral rib 52 and for being received in the peripheral recess 53 of the housing 50 and for detachably securing the needle support 60 in the front portion of the housing 50.

The peripheral swelling 631 of the needle support 60 is allowed to be moved over the peripheral rib 52 of the housing 50 again, for allowing the peripheral swelling 631 of the needle support 60 to be disengaged from the peripheral recess 53 of the housing 50 and for allowing the needle support 60 to be engaged into the housing 50 (FIGS. 6, 7) after use.

The needle support 60 further includes one or more projections 632 extended radially and outwardly from the peripheral swelling 631 of the needle support 60, for allowing the needle support 60 or the peripheral swelling 631 to be in point-contact with the housing 50, and for allowing the peripheral swelling 631 to be easily moved or engaged over the peripheral rib 52 of the housing 50.

As best shown in FIGS. 2 and 5-7, the projections 632 include a thickness less than that of the peripheral rib 52, such that the peripheral swelling 631 may also be engaged between the peripheral shoulder 51 and the peripheral rib 52 of the housing 50 when the projections 632 and the peripheral swelling 631 are engaged in the peripheral recess 53 of the housing 50.

The syringe housing 50 includes a cone-shaped or an inclined front portion 58 for engaging with the needle support 60 and for preventing the needle support 60 from being moved outwardly through the front portion of the housing 50, and for preventing the needle support 60 from being disengaged from the housing 50.

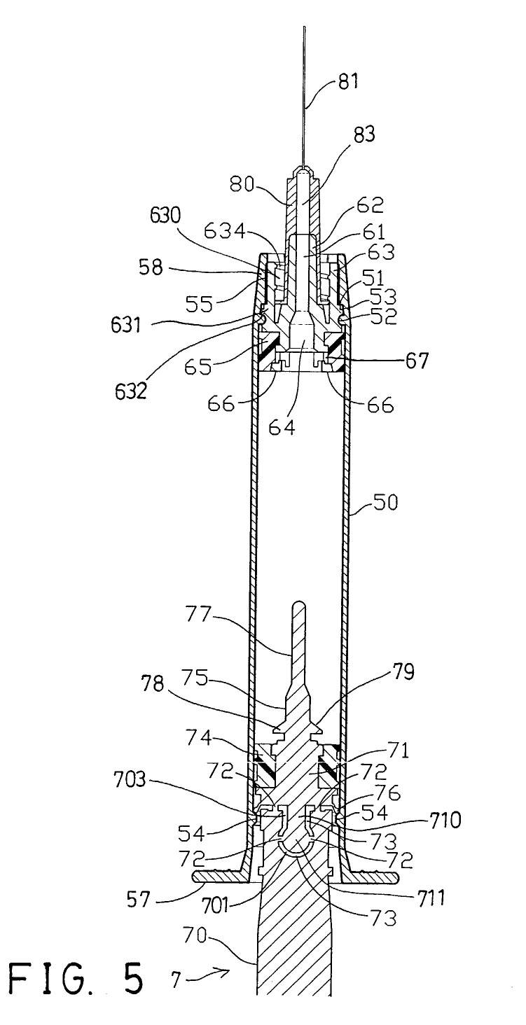

The needle support 60 includes a tube 62 extended forwardly therefrom for attaching or securing a barrel 80 thereto, and includes a bore 61 formed therein, such as also formed through the tube 62, and includes a chamber 64 (FIG. 5) formed in the rear portion thereof and communicating with the bore 61 thereof. The barrel 80 is provided for securing a needle 81 thereto and extendible outwardly through the housing 50, and includes a passage 83 formed therein and communicating with the bore 61 of the needle support 60.

The needle support 60 includes a peripheral wall 63 extended forwardly therefrom and provided or disposed around the tube 62, for forming or defining a peripheral space 630 between the peripheral wall 63 and the tube 62. The peripheral wall 63 includes one or more grooves 633 formed therein thereof and communicating with the peripheral space 630 thereof for forming or defining one or more blades 639 and for increasing the resilience of the peripheral wall 63.

An inner thread 634 is formed in the inner peripheral portion of the peripheral wall 63, for threading with the barrel 80, and for allowing the barrel 80 and thus the needle 81 to be secured to the tube 62 of the needle support 60.

The housing 50 includes one or more juts 55 provided in and extended inwardly from the front portion thereof. The juts 55 each includes a wider outer end 551 and a sharper inner end 553 which directed or faced toward the rear end or the panel 57 of the housing 50, for forming a triangular or wedge shape.

The needle support 60 also includes one or more bulges 635 provided on and extended outwardly from the outer peripheral portion of the peripheral wall 63. The bulges 635 each includes a wider outer end 637 directed or faced toward the peripheral swelling 631, and a sharper inner end 638 which directed or faced away from the peripheral swelling 631, for forming a triangular or wedge shape.

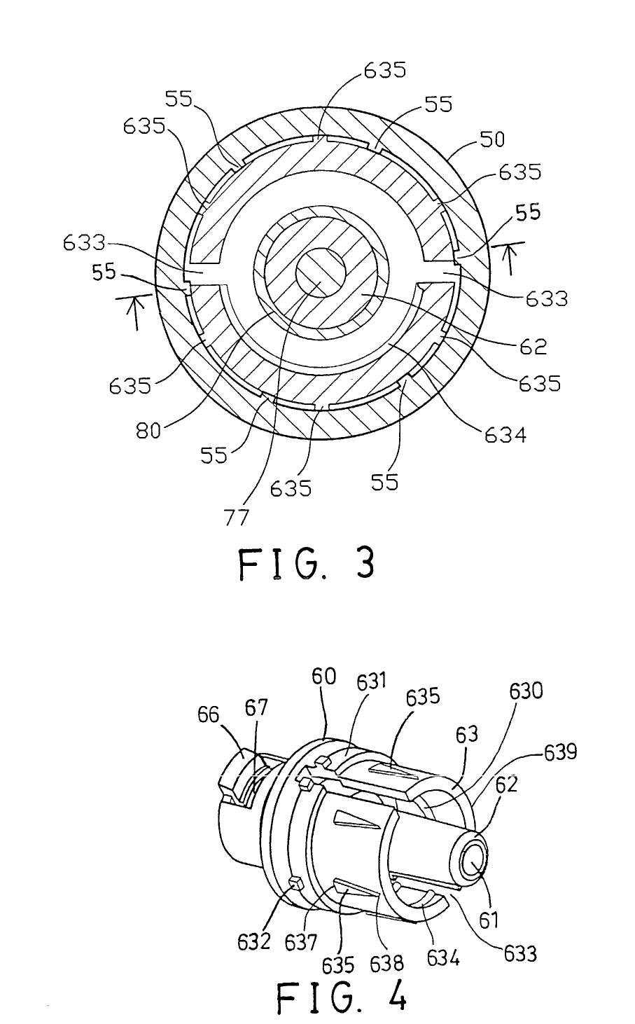

The sharper ends 638 of the bulges 635 and the sharper ends 553 of the juts 55 are arranged for allowing the bulges 635 of the needle support 60 to be easily engaged into the spaces formed or defined between the juts 55 of the housing 50 (FIG. 3), and thus for limiting the rotational movement between the needle support 60 and the housing 50, and thus for allowing the barrel 80 to be easily rotated or threaded relative to the needle support 60.

A sealing ring 65 is engaged on the rear portion of the needle support 60 and engaged between the needle support 60 and the housing 50 for making a water tight seal between the needle support 60 and the housing 50. The needle support 60 further includes one or more, such as two latches 66 extended on the rear portion thereof, and a notch 67 formed between the latches 66 and the needle support 60 respectively (FIGS. 4, 5).

A plunger 7 includes a piston 71 provided in the front portion thereof, such as provided in front of a stem 70, and a sealing ring 74 engaged on or around the piston 71, and slidably engaged and received in the housing 50 for making a water tight seal between the plunger 7 and the housing 50.

The plunger 7 further includes a rod 75 extended forwardly from the piston 71, and a pole 77 further extended forwardly from the front portion of the rod 75, for being slidably engaged or received in the chamber 64 and the bore 61 of the needle support 60 respectively, for adequately forcing or injecting the medicine out of the needle support 60 (FIGS. 2, 6, 7).

The plunger 7 includes a peripheral rib 78 extended radially and outwardly from the piston 71 thereof or from the rod 75, and having a tapered or inclined front surface 79 formed thereon (FIGS. 1, 5), for allowing the peripheral rib 78 to be moved over the latches 66 and to be engaged into the notches 67 of the needle support 60 (FIGS. 2, 6, 7), and thus for securing the piston 71 of the plunger 7 to the needle support 60.

The plunger 7 further includes one or more spring catches 76 extended laterally and outwardly therefrom, and provided for moving beyond the peripheral flange 54 of the housing 50 when the piston 71 is moved toward the needle support 60, and may be prevented from being moved rearward or backward beyond the peripheral flange 54 of the housing 50 after the piston 71 has been moved into the housing 50 and has been moved toward the needle support 60 (FIG. 7).

As shown in FIGS. 2, 5-7, the piston 71 includes an extension 710 extended rearwardly from the piston 61 and an enlarged head 711 formed on the rear portion of the extension 710. The stem 70 of the plunger 7 includes an opening 701 and a channel 703 formed in the front portion thereof for receiving the head 711 and the extension 710 of the piston 71 respectively, and for forming or defining a gap or a slot 73 between the stem 70 and the piston 71.

The plunger 7 includes one or more coupling members 72 formed or coupled between the stem 70 and the piston 71 and/or the extension 710 or the head 711 of the piston 71, for allowing the stem 70 to be bent or broken or disengaged from the piston 71 (FIG. 7) when the stem 70 is bent relative to the piston 71.

In operation, as shown in FIG. 5, the medicine received in the housing 50 may be forced and injected out through the needle 81 by the piston 71 when the piston 71 is moved and forced toward the needle support 60 (FIG. 2). The peripheral rib 78 of the plunger 7 may be moved over the latches 66 and engaged into the notches 67 of the needle support 60 (FIGS. 2, 6, 7), for securing the piston 71 to the needle support 60.

When the stem 70 of the plunger 7 is pulled rearwardly, the needle support 60 and the needle 81 may be pulled into the housing 50 (FIGS. 6, 7). The stem 70 may then be bent relative to the piston 71 (FIG. 7) such that the needle support 60 and the needle 81 may be retained in the housing 50 and may be prevented from being used again after use.

As shown in FIG. 5, when the peripheral rib 78 of the plunger 7 has not been engaged into the notches 67 of the needle support 60 and has not been secured to the needle support 60, and when the stem 70 has not been bent relative to the piston 71 and disengaged from the piston 71, the piston 71 may also be used and moved away from the needle support 60 for drawing the blood or the like into the housing 50.

Accordingly, the syringe in accordance with the present invention includes a needle safely receiving structure for safely receiving the needle after use and for preventing the syringe from being used again after use, and includes a stably retained needle support for allowing the needle to be easily threaded or rotated relative to the needle support and to be attached to the syringe housing, and for allowing the medicine to be adequately forced or injected outward of the syringe housing and the needle support, and for allowing the amount of the residual medicine to be smaller than 0.075 ml.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A syringe comprising:
a housing including a front portion and a rear portion,
a needle support received in said front portion of said housing, and including a front portion having a tube extended therefrom, and including a bore formed therein,
a needle attached to said tube of said needle support and extendible outward of said housing,
a plunger including a piston slidably received in said housing and movable toward and away from said needle support, and including a stem detachably secured to said piston, for moving said piston toward and away from said needle support,
wherein said needle support includes a rear portion having at least one spring latch provided thereon, and a notch formed between said at least one spring latch and said needle support, said piston includes a front portion having a peripheral rib laterally extended therefrom for moving over said at least one spring latch and for engaging into said notch of said needle support, and for securing said piston to said needle support when said piston is forced toward said needle support, said needle support is movable into said housing by said plunger when said piston of said plunger is moved away from said front portion of said housing, and
said housing includes at least one jut provided in said front portion thereof, said needle support includes at least one bulge extended therefrom for engaging with said at least one jut of said housing, and for limiting a rotational movement between said needle support and said housing.

2. The syringe according to claim 1, wherein said at least one jut of said housing includes a first end located at said front portion of said housing, and includes a second end directed toward said rear portion of said housing and sharper than said first end of said at least one jut.

3. The syringe according to claim 1, wherein said needle support includes a rear portion, said at least one bulge of said needle support includes a first end located at said front portion of said needle support, and includes a second end directed toward said rear portion of said needle support and sharper than said first end of said at least one bulge.

4. The syringe according to claim 3, wherein said needle support includes a peripheral wall provided on said front portion thereof and provided and formed around said tube for forming a peripheral space between said tube and said peripheral wall, said at least one jut is provided on said peripheral wall.

5. The syringe according to claim 4, wherein said peripheral wall includes at least one groove formed therein for increasing a resilience of said peripheral wall.

6. The syringe according to claim 1, wherein said housing includes an inner peripheral recess formed in said front portion thereof and defined by a peripheral rib, said needle support includes a peripheral swelling extended outwardly therefrom for moving over said peripheral rib and for engaging into said peripheral recess of said housing, and for detachably securing said needle support to said housing.

7. The syringe according to claim 6, wherein said needle support includes at least one projection extended from said peripheral swelling for engaging into said peripheral recess of said housing.

8. The syringe according to claim 1, wherein said needle support includes a rear portion having a chamber formed therein and communicating with said bore thereof, said piston includes a rod and a pole extended forwardly therefrom for engaging into said chamber and said bore of said needle support respectively.

9. The syringe according to claim 1, wherein said piston includes a rear portion having an extension extended therefrom, said stem includes a front portion having a channel formed therein for receiving said extension of said piston, and for defining a slot between said stem and said extension of said piston, and at least one coupling member formed and coupled between said stem and said extension of said piston, for detachably securing said stem to said piston.

10. The syringe according to claim 9, wherein said piston includes a head formed on said extension thereof, said stem includes an opening formed therein and communicating with said channel thereof for receiving said head, and includes at least one coupling member formed and coupled between the stem and said head of said piston.
